(19) United States
(12) Patent Application Publication
(10) Pub. No.: US 2003/0228335 A1
(43) Pub. Date: Dec. 11, 2003

(54) Method for improving sensory characteristics of semisolid predominantly anhydrous lipids

(76) Inventor: Hans R. Suess, Starrkirch-Wil (CH)

Correspondence Address: Reed Smith LLP, 2500 One Liberty Place, 1650 Market Street, Philadelphia, PA, 19103-7301, US

(21) Appl. No.: 10/163,523

(22) Filed: Jun. 6, 2002

(51) Int. Cl. A61K 7/42

(52) U.S. Cl. 424/401

(57) ABSTRACT

A composition comprises amphiphobic halocarbon particles, and amphiphilic lipids having at least 2 hydroxyl groups. This composition may be used in methods of decreasing oiliness, greasiness, stickiness and tackiness of anhydrous semisolid lipids, methods of delivering pharmaceutical active ingredients, methods of preventing or treating sunburn or ultraviolet radiation, methods or preventing or treating skin disorders and methods of delivering cosmetics.

### BACKGROUND OF THE INVENTION

The present invention is directed to a method of improving the adhesion of semisolid predominantly anhydrous lipids to certain substrates. This method decreases oiliness, greasiness, stickiness, and tackiness and improves the adhesion of the lipids to a substrate. These lipids may be applied to surfaces, including, but not limited to hair, leather, paper, skins textiles and wood, where an impermeableness against water, but not water vapor, is desirable.

The principal function of the skin is to keep water in and exogenous substances out. This is accomplished by a highly ordered barrier, located in the stratum corneum, the skin's outermost layer. The stratum corneum composed of corneocytes (or horny cells) embedded in lipid lamellae, composed mainly of ceramides, cholesterol and free fatty acids. The horny layer is incessantly exposed to an oxidative environment (including, but not limited to UV radiation, ozone, aerobic microorganisms, and metallic microparticles). However, the horny layer is defended by antioxidant tocopherols, which are contained in the sebum that is continually secreted by the sebaceous glands, and ascorbic acid, which is secreted by the eccrine glands.

By frequent washing, especially of the hands, sebum and eccrine fluid is withdrawn leaving the skin exposed to increased oxidative stress, which damages a linoleic acid containing ceramide. This may result in a decreased barrier function and therefore an increased transepidermal water loss, which may ultimately cause dry skin.

Anhydrous lipid-rich preparations such as petrolatum or lanolin, which are not easily washed-off, seem appealing but they are not used since they are lacking in cosmetic elegance. Therefore, less greasy skin creams are often applied. Such skin creams are often emulsions containing emulsifiers which are known to be potential skin irritants. Additionally, such emulsions contain a water phase which has to be preserved in order to avoid microbial contamination. Perfumes are often included in these formulations. Both the preservatives and perfumes frequently cause allergic reactions.

In the formulation of dermatological vehicles, therapeutic efficacy is an important goal. Skin penetration of active ingredients are frequently more enhanced by anhydrous semisolid excipients than by creams (which are semisolid emulsions). Anhydrous semi-solid excipients based on hydrocarbons moisturize the skin by occlusion and enhance overall skin quality more readily than creams. Furthermore, anhydrous semi-solid hydrocarbon excipients do not degrade from bacterial lipases. In spite of these advantages, creams are very often preferred to ointments, in the art, because they are more pleasant to use and therefor better accepted by patients.

On the other hand there is a disadvantage of semisolid anhydrous ointments as moisturizers. If they are applied immediately prior to dressing, and only a short time is available for absorption, then a noteworthy excess residue of the applied product is left to be absorbed by clothes, possibly affecting the actual use and acceptability.

Prior patents provide no teaching concerning the use of combinations of amphiphobic submicro or microparticles of halocarbons and amphiphilic diols, triols or polyols as effective in improving the cosmetic elegance of semisolid lipids. An amphiphilic product includes both hydrophilic and lipophilic groups. An amphiphobic product does not include either a hydrophilic or a lipophilic group.

Other patents in this field include British Patent 1575 201 (filed Oct. 22, 1975); U.S. Pat. No. 4,087,517 (1976); Japanese Patent No. 6228 3920; European Patent No. 0 598 412; and WO 02/03946 A1.

The greasier and stickier a moisturizer is, the less cosmetically acceptable it is. Increases in the lipid content of a moisturizer often increases the greasiness and stickiness of the moisturizer. For this reason, it is desirable to suppress the greasiness and stickiness of lipid-rich preparations (e.g. petrolatum or other formulations consisting mainly of hydrocarbons). As was recognized by Kligman twenty years ago, “A boon to all sufferers from dry skin would be a product with the efficacy of petrolatum and the hedonic-aesthetic properties of a light oil in water cream.” (Kligman A. M. and Leyden J. J.: Safety and Efficacy of Topical Drugs and Cosmetics, New York Grune & Stratton, 1982, p. 236.)

Water is the plasticiser of the skin. Lipid content is of major importance to the plasticity of the skin because the presence of lipids decreases transepidermal water loss and consequently increases moisture content of the stratum corneum. Therefore, lipid-rich formulations are effective in the treatment of dry skin.

### SUMMARY OF THE INVENTION

The present invention is quite unexpected. The sensory characteristics, the aesthetic appeal and thereby the user acceptance of anhydrous semisolid lipids can be greatly improved with the addition of a combination of amphiphobic micro or sub-micro particles of polymerized perhalogenoalkenes and at least one amphiphilic lipid containing at least two hydroxyl groups.

Examples of such semisolid lipids are mainly complex mixtures of hydrocarbons (e.g., without limitation, petrolatum) or combinations of liquid hydrocarbons (e.g., without limitation, paraffin oils or oligomers of monoolefins, e.g., without limitation, ethylene, propylene, butylene, isobutylene, and decene) with solid polymers of monoolefins, esters and combinations of liquid and/or solid esters (e.g., without limitation, waxes), semisoft triglycerides or any combination thereof (e.g., without limitation, lanolin or wool wax).

A preferred embodiment is anhydrous lanolin, a water-free refined substance, which is a rather complex mixture of esters of alcohols (e.g., without limitation, monohydric, dihydric, sterols, and triterpenols) with straight or branched fatty acids.

The lipids, used in this invention, preferably contain minimal quantities of water. The amount depends on the chemical composition. For example, centrifuged raw wool wax contains 1-5% water. At 32° C. (the approximate temperature of the skin surface), water is only very slightly soluble in liquid hydrocarbons (less than 75 ppm), whereas esters (e.g., without limitation, dibutyladipat) may contain up to 100 times more water.

Lipids with high water content are less occlusive than lipids with low water content. For example, hydrocarbons, which have a low water content, excel in the treatment of dry skin by occlusion.

Branched lipids (e.g., without limitation, petrolatum) are generally sticky and greasy. Additionally, bulky lipids (e.g., without limitation, anhydrous lanolin) also tend to be sticky and greasy. On the other hand, straight waxes (e.g., without limitation, jojoba oil or mixtures with other waxes, e.g., without limitation, beeswax, carnauba, or candelilla wax) are much less greasy and sticky. However, patient compliance may be improved by the present invention because the skin feel is further improved.

The dry powders of such micro or sub-micro particles may be agglomerated. In which case, the particles have to be de-agglomerated in order to obtain an even distribution of the particles in the final composition. This can be accomplished by using volatile solvents which show some affinity to the halopolymer. Preferred solvents include, but are not limited to, volatile silicones, linear or cyclic, having from 2 to 7 silicon atoms and from 1 to 10 alkyl groups (e.g., without limitation, hexamethyldisiloxane, dimethicones, octamethylcyclotetrasiloxane, decamethylcyclopenta-siloxane, dimethicone siloxane, octamethyltrisiloxane and/or mixtures thereof). Perfluorobutyl ether and methylperfluoroisobutylether (e.g., without limitation, BFE-7100 and HFE-7200 from 3M Chemicals), which show a high affinity to the halogenopolymers, are preferred solvents. Other preferred solvents are C₈₋₉ to C₁₂₋₂₀ isoparaffins (e.g., without limitation, Isopar® from Exxon Corporation). The solvents may be used individually or in combination.

The submicro or micro particles of the perhalogenopolymers may be dispersed in a convenient liquid. Micronised or submicronized perhalopolymers have been known in the art for more than a quarter of a century. However, until the present invention, they did not work as expected in the field of skin protectants and skin care because semisolid anhydrous preparations containing such particles lack cosmetic elegance. Therefore, the compliance of treatments with such preparations was unsatisfactory. Additionally, when the micronized or submicronized particles are used as a component of an emulsion, they lose a great part of their sensorial qualities (often from interactions with emulsifiers).

In an anhydrous semisolid lipid composition of the present invention, glycerol would not be soluble at 3%. In one embodiment, the solubility parameters of cyclomethicones, e.g., without limitation, white mineral oil and petrolatum are in the 5.77 to 7.33 range, whereas those of propylene glycol is 14.0 and glycerol is 16.26. A difference of smaller than 2 usually indicates mutual solubility (Vaughan C. D., J. Soc. Cosmet. Chem. 36, 319-333, 1985).

Examples of suitable diols/polyols in accordance with the present invention for improving the sensory properties of semisolid embodiments are lipid-soluble molecules containing 2 or more hydroxyls, including, but not limited to, dipropylene glycol, 2-ethylhexanediol, octanediol, panthenol, phytantriol, certain ceramides (6), and sphinganine.

Lipoinsoluble polyols (e.g., without limitation, inositol or glycerol) can be made liposoluble by etherification or esterification with a suitable alcohol or acid respectively. The molecular weight of such components is below 1000 and the liposolubility has to be at least 0.2%w/w. Examples are, without limitation, glycerolisostearate, pentaerithityl mono- and distearate (which are emulsifiers). These example may interfere with the stratum corneum lipids. More preferable examples are sphingosine and (soy-) lecithin (which contains phosphatidyl inositol).

Suitable halocarbon microparticles have diameters in the range of 0.1 (or smaller) to 50 micrometers, such as Algoflon HC® from Ausimont, FluoroPUREr from Shamrock Technologies, and Fluortress from DuPont. See, International Cosmetic Ingredients Dictionary and Handbook, 8th Ed 2000. The diameter of micro or sub-micro particles is preferably about 100 nanometers to about 50 microns.

### DETAILED DESCRIPTION OF THE INVENTION

Compositions of the present invention with their superior skin feel are ideal excipients for dermatological products since they adhere well to skin, and are not easily washed off. For these reasons, active ingredients, delivered by these excipients, remain in contact with the skin longer. In most cases, an increased occlusivity induces a higher moisturization of the skin, and as a consequence, an increased transdermal absorption of active ingredients. By varying the type and amount of amphiphilic bis/oligo hydroxylated lipids, the occlusive effect can be varied correspondingly.

Many formulations have been made in laboratory experiments to show the benefits of the various embodiments of the invention and the ingredients used.

### EXAMPLE 1

| Weight % | |
|---|---|
| 74.0 | Mineral oil (viscosity 240 mm²/s) |
| 3.0 | Jojoba oil |
| 10.0 | Polyethylene (A-C ® polyethylene 617 |
| | from Allied Signal) |
| 1.5 | Panthenol |
| 1.5 | Polytetrafluoroethylene (“PTFE”) (Fluoro |
| | 4 from Shamrock) |
| 10.0 | Octamethylcyclotetrasiloxane |

Heat mineral oil, jojoba oil, A-C polyethylene and panthenol in a container equipped with a stirrer to 95° C. When a homogenous liquid is obtained, add polytetrafluoroethylene (PTFE) dispersed in the octamethylcyclotetrasiloxane. Cool under continuous stirring to 50° C.

All percentages in this disclosure are weight percentages unless otherwise noted.

### EXAMPLE 2

| Weight % | |
|---|---|
| 61.0 | Anhydrous lanolin USP |
| 7.4 | A-C Polyethylene 617 A |
| 1.7 | Ceramide VI (Gist-Brocades ® from |
| | Cosmoferm) |
| 2.1 | PTFE (Fluorotress Powder from DuPont) |
| 27.8 | Decamethylcyclopentasiloxane |

### EXAMPLE 3

| Weight % | |
|---|---|
| 70.0 | Hydrogenated Poly-1-decene (2008 FG |
| | from Neste) |
| 11.0 | Polyethylene (Siltek ® PL Polymer from |
| | Petrolite Corporation) |
| 1.9 | PTFE (Algoflor AC from Ausimont) |
| 2.8 | 1,8-Octanediol |
| 14.3 | Dimethicone (and) Octamethyltrisiloxane |
| | (Dow Corning 2-1184) |

### EXAMPLE 4

| Weight % | |
|---|---|
| 62.2 | Hydrogentated Polyiosbutene (Panalene |
| | L-14 from Amoco Chemical) |
| 12.0 | Polyethylene (Hoechst Wax PE 520 from |
| | Clariant) |
| 2.0 | Anhydrous lanolin |
| 2.8 | 2-ethyl-1,3-hexanediol (EHDIOL from |
| | Dixie Chemical Co.) |
| 7.0 | PTFE (submicron) in Polyisobutene |
| | (Fluoropure Ultra Fine 25 cs from |
| | Shamrock) |
| 14.0 | Dimethicone (Belsil DM 1 plus from |
| | Wacker) |

Heat panalene, Hoechst wax, lanolin and EHDIOL to 115° C. until a clear solution is obtained, then add the dispersed PTFE and silicone and cool while stirring to 40° C.

In this example, the amount of polyethylene may be reduced in order to obtain a viscous liquid. The nanoparticle size of the used PTFE induces practically no sedimentation.

### EXAMPLE 5

| Weight % | |
|---|---|
| 78.0 | Geahlene 1600 (from Penreco) |
| 2.0 | Jojoba oil |
| 2.0 | Beeswax |
| 2.3 | Lecithin NF (contains inositol |
| | phosphatide) |
| 2.3 | PTFE micronized |
| 13.4 | Hexamethyldisiloxane |

Heat the ingredients, except the PTFE and the siloxane, to 80° until a solution is obtained. Then cool to 60° C. and add the remaining components. Because hexamethylsiloxane is very volatile, it is preferable to use a closed kettle equipped with a condenser.

### EXAMPLE 6

| Weight % | |
|---|---|
| 45.0 | Petrolatum USP |
| 20.0 | Ozokerite |
| 2.4 | Phytantiol |
| 2.6 | PTFE micronized |
| 20.0 | C₁₁₋₁₃ isoparaffin (from Exxon Chemical) |
| 10.0 | Perfluorobutylether and |
| | perfluoroisobutylether (HFE-7200 from 3M) |

Add PTFE dispersed in HFE-7200 together with the isoparaffin to the molten other ingredients.

It is understood that while the invention has been described in conjunction with the detailed description thereof, that the foregoing description is intended to illustrate and not limit the scope of the invention, which is defined by the scope of the appended claims. Other aspects, advantages, and modifications are evident from a review of the following claims.

What is claimed:

1. A method of decreasing oiliness, greasiness, stickiness and tackiness of anhydrous semisolid lipids comprising combining amphiphobic halocarbon particles and at least one amphiphilic lipid having at least 2 hydroxyl groups.

2. The method of claim 1 wherein the amphiphobic halocarbon particles are selected from the group consisting of polytetrafluorethylene, polychlorotrifluoroethylene, polyhexafluoropropylene, polyvinylidine fluoride, polyvinylfluoride, copolymer mixtures of tetrafluorethylene and ethylene, and mixtures of tetrafluoroethylene, propylene and fluorinated copolymers of ethylene propylene.

3. The method of claim 2 wherein the amphiphobic halocarbon particles are present at about 0.2% to about 20%.

4. The method of claim 2 wherein the amphiphobic halocarbon particles are present at about 0.5% to about 5%.

5. The method of claim 2 wherein the amphiphobic halocarbon particles are present at about 1.5% to about 2.5%.

6. The method of claim 1 wherein the amphiphilic lipids are selected from the group consisting of diols, thiols and polyols.

7. The method of claim 6 wherein the amphiphilic lipids are soluble to at least 0.2% in an anhydrous excipient and the lipids have a molecular weight of less than 1000.

8. The method of claim 1 wherein the amphiphobic halocarbon particles are in solution, the solution comprises a volatile solvent.

9. The method of claim 8 wherein the solvent has a boiling point below 250° C.

10. The method of claim 8 wherein the solvent is selected from the group consisting of hexamethyldisiloxane octamethylcyclotetrasiloxane, decamethylcyclopentasiloxane, dimethicone and octamethyltrisiloxane, dimethicone and mixtures thereof.

11. The method of claim 8 wherein the solvent is selected from the group consisting of C₁₁₋₁₃ isoparaffin.

12. The method of claim 11 wherein the paraffin is present at about 0% to about 60%.

13. A method of delivering pharmaceutical active ingredients comprising combining amphiphobic halocarbon particles and at least one amphiphilic lipid having at least 2 hydroxyl groups and adding at least one pharmaceutical active ingredient to the combination.

14. A method of preventing or treating sunburn or ultraviolet radiation comprising topically applying a combination of one or more sunscreens, amphiphobic halocarbon particles and at least one amphiphilic lipid having at least 2 hydroxyl groups.

15. A method of preventing or treating skin disorders comprising topically applying amphiphobic halocarbon particles and at least one amphiphilic lipid having at least 2 hydroxyl groups.

16. The method of claim 15 wherein the skin disorder is dry skin.

17. A method of delivering cosmetics to skin comprising topically applying a combination of one or more cosmetics, amphiphobic halocarbon particles and at least one amphiphilic lipid having at least 2 hydroxyl groups.

18. A composition comprising amphiphobic halocarbon particles; and amphiphilic lipids having at least 2 hydroxyl groups.

19. The composition of claim 18 further comprising one or more pharmaceutically active ingredients.

20. The composition of claim 18 further comprising one or more sunscreens.

21. The composition of claim 18 further comprising one or more cosmetic ingredients.

22. The composition of claim 18 wherein the amphiphobic halocarbon particles are selected from the group consisting of polytetrafluorethylene (PTFE), polychlorotrifluoroethylene, polyhexafluoropropylene, polyvinylidine fluoride, polyvinylfluoride, copolymer mixtures of tetrafluorethylene and ethylene, and mixtures of tetrafluoroethylene, propylene and fluorinated copolymers of ethylene propylene.

23. The composition of claim 22 wherein the amphiphobic halocarbon particles are present at about 0.2% to about 20%.

24. The composition of claim 22 wherein the amphiphobic halocarbon particles are present at about 0.5% to about 5%.

25. The composition of claim 22 wherein the amphiphobic halocarbon particles are present at about 1.5% to about 2.5%.

26. The composition of claim 18 wherein the amphiphilic lipids are selected from the group consisting of diols, thiols and polyols.

27. The composition of claim 26 wherein the amphiphilic lipids are soluble to at least 0.2% in an anhydrous excipient and the lipids have a molecular weight of less than 1000.

28. The composition of claim 18 wherein the amphiphobic halocarbon particles are in solution, the solution comprises a volatile solvent.

29. The composition of claim 28 wherein the solvent has a boiling point below 250° C.

30. The composition of claim 28 wherein the solvent is selected from the group consisting of hexamethyldisiloxane octamethylcyclotetrasiloxane, decamethylcyclopentasiloxane, dimethicone and octamethyltrisiloxane, dimethicone and mixtures thereof.

31. The composition of claim 28 wherein the solvent is selected from the group consisting of C₁₁₋₁₃ isoparaffin.

32. The composition of claim 31 wherein the paraffin is present at about 0% to about 60%.
